(19) United States
(12) Patent Application Publication
(10) Pub. No.: US 2014/0358203 A1
(43) Pub. Date: Dec. 4, 2014

(54) Thermal Assistance Device

(71) Applicant: Justin Li, Dallas, TX (US)

(72) Inventor: Justin Li, Dallas, TX (US)

(21) Appl. No.: 13/904,329

(22) Filed: May 29, 2013

(51) Int. Cl. A61F 7/00

(52) U.S. Cl. CPC A61F 7/00
USPC 607/108; 607/112

(57) ABSTRACT

A thermal assistance device is useful to provide supplemental heating or cooling to a user's circulating core blood, or to the blood supply to an extremity of a user.

### BACKGROUND OF THE INVENTION

1. Field of the Invention

The present invention relates to a device useful to provide supplemental heating or cooling to a person's circulating core blood. The device may be configured as a garment, or as a band, strap, or wrap, for example, to be worn on a wrist, arm, or leg. The thermal assistance device may also be configured in a variety of other forms. The inventive device or garment may also be used to provide supplemental heating or cooling to the blood supply to a person's extremity (i.e., to a hand or foot, for example).

2. Related Technology

People involved in strenuous activities, especially in hot climates, or possibly in protective or restrictive clothing and apparel are at risk of overheating (hyperthermia). Overheating reduces a person's mental faculties, reduces stamina, and generally compromises physical, and mental faculties, and the ability to accomplish tasks. On the other hand, people in cold conditions are at risk of hypothermia. Again, hypothermia reduces a person's physical stamina, compromises mental faculties, and reduces the ability to accomplish tasks. An over-arching consideration is that human beings have a limited range of working temperatures in which they can function effectively. Both high or low ambient temperature conditions, heavy or restrictive clothing, heavy physical exertions, or the task of accomplishing complex or demanding tasks, all can contribute to a too high or too low of a core body temperature. People who experience either too high or too low a core body temperature experience compromised performance.

Human extremities (i.e., the hands and feet, especially if exposed) are susceptible to loss of function or dexterity as a result of environmental cold, especially in conditions of extreme cold or conditions of rapid heat loss from the hands, such as immersion in water. Under such conditions if a person's hands lose too much heat and become chilled the person loses hand dexterity, strength, and precision of movement. Similarly, if a person's feet lose too much heat in extreme conditions frostbite of the toes and feet, and catastrophic damage or loss of the toes or feet can result.

Historically, people have attempted to deal with excessive heat by expedients such as forehead absorptive or cooling bands, damp handkerchiefs about the neck, and similar. Excessive cold has been dealt with by gloves, insulted boots, catalytic combustion hand warmers, and similar. None of these historical expedients has been entirely satisfactory.

### SUMMARY OF THE INVENTION

Accordingly, one particularly preferred embodiment of the present invention provides a thermal assistance device comprising an especially configured holder disposed at a selected location on a wearer's body. The especially configured holder is configured for receiving and disposing a heating or cooling device in an abiding heat transfer relation with the wearer's skin at the selected location. The especially configured holder is disposed at a selected location where there is a superficially exposed, or near-superficially exposed (i.e., a pulse point) artery, and mild compression is maintained between the heating or cooling device and the wearer's skin, so as a result an effective heat transfer relationship with the wearer's circulating blood is established and maintained between the heating or cooling device and the person's circulating blood.

Other objects, features, and advantages of the present invention will be apparent to those skilled in the art from a consideration of the following detailed description of a preferred exemplary embodiment thereof taken in conjunction with the associated figures which will first be described briefly.

This invention relates to such a thermal assistance device which has an especially configured pocket, holder, or receptacle disposed at a selected location of a wearer's body. A heating or cooling device held by the device is disposed in an abiding heat transfer relation of mild compression with the person's skin, and in a resulting effective heat transfer relationship with the person's circulating blood in superficially exposed, or near-superficially exposed (i.e., a pulse point) artery. Preferably, both the especially configured pocket, holder, or receptacle, and the heating or cooling device are both elongate, are disposed over the superficially exposed artery, and are aligned along their length with this artery. By providing supplemental cooling or heating of the circulating blood, the person's core body temperature can be moderated toward a desired temperature range. Alternatively, supplemental heating or cooling to a person's extremity may be accomplished.

### BRIEF DESCRIPTION OF THE DRAWING FIGURES

FIG. 1 is a diagrammatic view of a person wearing a garment embodying the present invention;

FIG. 2 is a fragmentary cross sectional view taken along line 2-2 of FIG. 1, and viewing in the direction of the arrows;

FIG. 3 is a fragmentary cross sectional view taken along line 3-3 of FIG. 1, and viewing in the direction of the arrows;

FIG. 4 is a diagrammatic view of a person wearing an alternative embodiment of a garment embodying the present invention;

FIG. 5 is a diagrammatic view similar to FIG. 1 but showing a much smaller portion of a person wearing an alternative embodiment of a garment embodying the present invention;

FIG. 6 is a fragmentary cross sectional view taken along line 6-6 of FIG. 5, and viewing in the direction of the arrows; and

FIG. 7 is a fragmentary view of still another alternative embodiment of the present invention, configured as a strap, band, or wrap to be worn on the wrist, arm or leg of a user.

### DETAILED DESCRIPTION OF EXEMPLARY PREFERRED EMBODIMENTS OF THE INVENTION

While the present invention may be embodied in many different forms, disclosed herein are several specific exemplary embodiments, which illustrate and explain the principles of the invention. In conjunction with the description of these embodiments, a method of providing supplemental heating and cooling to the circulating blood, or to an extremity of a person will be apparent. It should be emphasized that the present invention is not limited to the specific embodiments illustrated.

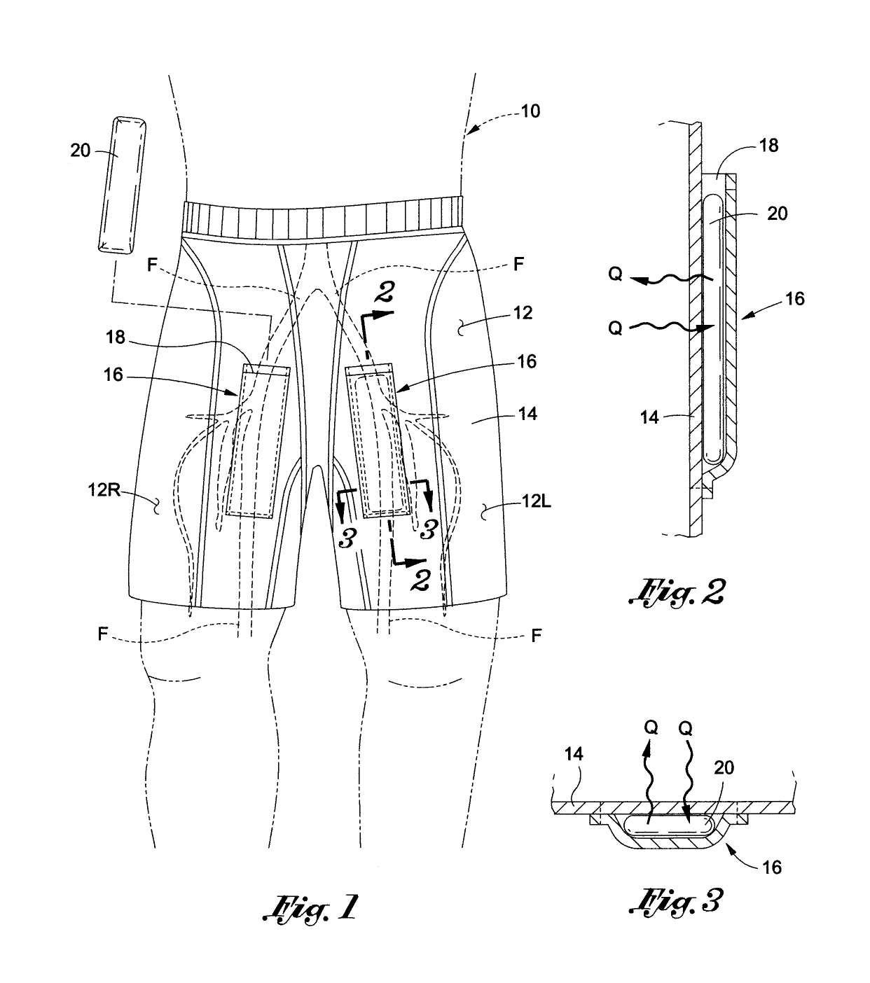

Viewing FIG. 1, a human 10 is depicted using or wearing a thermal assistance garment 12 embodying the present invention. The garment 12 in this embodiment is configured as a pair of compression shorts. Conventional versions of compression shorts will be familiar to joggers, bicycle riders, and others engaged in athletic pursuits. Accordingly, it is seen that the garment 12 includes an elastic outer shell of material 14, which forms the shorts 12, including a waist band 12A, and a left and right leg portion 12L and 12R, respectively. However, in contrast to the conventional compression shorts, the thermal assistance garment 12 according to this invention includes a pair of elongate generally vertically oriented pocket features, generally indicated with the arrowed numerals 16. The pockets 16 are disposed in this embodiment on the outside of the shell material 14, and each defines an upper opening 18. An elongate thermal pack or package 20 is received into each one of the pockets 16, as is indicated on the left-hand side of FIG. 1.

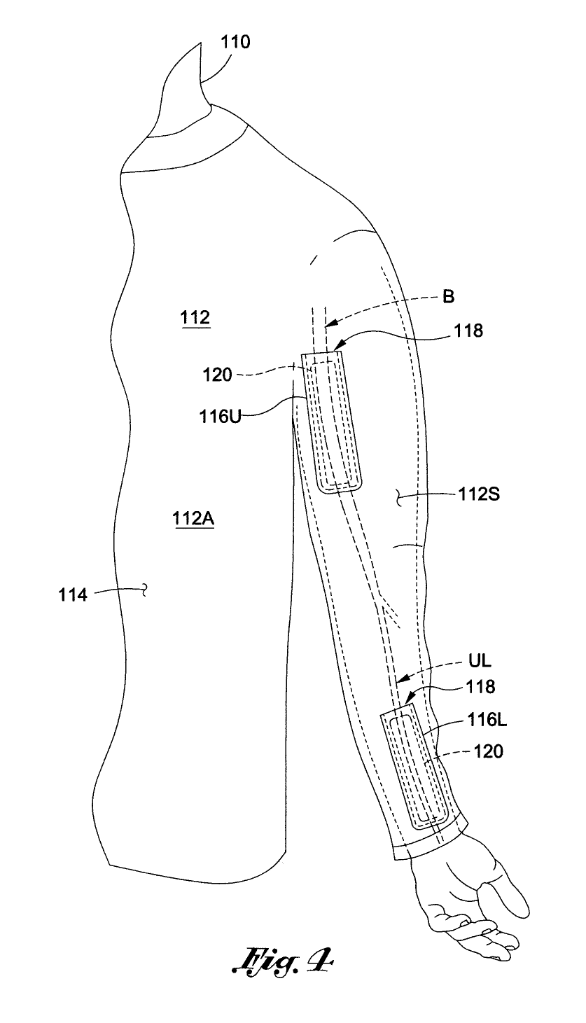

FIGS. 2 and 3 in conjunction indicate that because of the elasticity of the garment 12, the thermal packs 20 are disposed immediately adjacent to and in heat transfer relationship with the skin of the wearer 10, as is indicated by the arrowed characters “Q” on FIGS. 2 and 3. That is, the thermal packs 20 are separated from the skin of the user 10 by only the material 14 of the garment 12. The arrowed characters Q on FIGS. 2 and 3 are bi-directional (i.e., extend either toward or from the thermal pack 20) because the heat transfer so indicated can be heat flow either from the thermal pack 20 to the user 10 (i.e., supplying heat to the user), or heat flow from the user to the thermal pack 20 (i.e., cooling the user). The thermal packs 20 are active units which may be chemical reaction powered to either provide heat (i.e., exothermic chemical reaction) to the user 10, or to provide cooling (i.e., endothermic chemical reaction) to the user 10. The thermal packs 20 may provide heating or cooling by mechanisms other than chemical reaction. For example, the thermal packs may be thermoelectric, resistance, reverse Peltier effect, or any other desired form of heating or cooling device.

Importantly, the frontal view of user 10 illustrates that the garment 12 positions the thermal packs 20 over and in alignment along their length with the femoral arteries of the user 10. The femoral arteries are generally indicated by the dashed lines “F” on FIG. 1. Because of the elasticity of the garment 12 (i.e., providing mild compression), the thermal packs 20 are held by garment 12 in effective heat transfer relation with the user's skin, and with the user's blood circulating in the femoral arteries F. In this respect it may be considered important that the thermal packs 20 are elongate and are disposed over and in alignment with the femoral arteries F of the user 10. Consequently, heating or cooling provided by the thermal packs 20 is most effectively provided to either heat or cool the circulating core blood of the user 10 at the femoral arteries F.

Consequently, the thermal assistance garment 12, and thermal packs 20, can be utilized by a user to effectively providing cooling to the user's circulating core blood in conditions of extreme exertion or high temperatures, or while wearing protective apparel, for example. On the other hand, heating thermal packs 20 can be used along with garment 12 to assist the user in maintaining a safe core body temperature under frigid conditions.

Turning to FIG. 4, an alternative embodiment of a thermal assistance garment is illustrated being worn by a user or wearer 110. Because the garment seen in FIG. 4 is similar in many respects to that of FIGS. 1-3, features which are analogous in structure or function to those of FIGS. 1-3 are referenced on FIG. 4 with the same numeral used above, but increased by one hundred (100). Accordingly, attention to the garment 112 shown in FIG. 4 will reveal that the garment 112 is configured as a elastic compression shirt including a shirt body 112A (only a portion of which is seen in FIG. 4) fashioned of selectively elastic material 114, and a pair of sleeves 1125 also fashioned of selectively elastic material 114 (only one of which—the left sleeve—is seen in FIG. 4).

Accordingly, the shirt configuration of thermal assistance garment 112 seen in FIG. 4 according to this invention includes a pair of elongate pocket features, generally indicated with the arrowed numerals 116. The upper one 116U of this pair of pocket features 116 is disposed over and in alignment along its length with the brachial artery of the user's upper inner arm (indicated by dashed arrow B). On the other hand, the lower one 116L of this pair of pocket features 116 is disposed over and in alignment along its length with the ulnar/radial (i.e., inner wrist) artery of the user 110 (indicated by dashed arrow UL). The pockets 116U and 116L are both disposed in this embodiment on the outside of the shell material 114 of the shirt 112, and each defines a respective upper opening, generally indicated with the arrowed numeral 118. An elongate thermal pack or package 120 (i.e., like the thermal packs 20 discussed above) may be received into either one or both of the pockets 116U and 116L, as will by now be familiar to the reader.

In hot environmental conditions, for example, the wearer 110 may choose to employ a cooling thermal pack in the upper pocket 116U of both arms to assist in preventing overheating of the user's circulating core blood. An additional pair of cooling thermal packs can be used by the user in the lower pockets 116L for this purpose also. However, it will be understood that in cold weather conditions, or other conditions resulting in severe or rapid cooling of the hands, the user may choose to use warming packs in each of the pockets 116 U, 116L in order to provide supplemental warming to the user's core blood flow, and especially to provide warming of the blood flow to the user's hands. Such supplemental warming of the blood flow to an extremity (such as the user's hands, via the UL artery, as seen in FIG. 4) can be useful to the user in order to assist in maintaining hand strength and dexterity in cold weather conditions, or in conditions of rapid hand cooling, such as immersion in cold water.

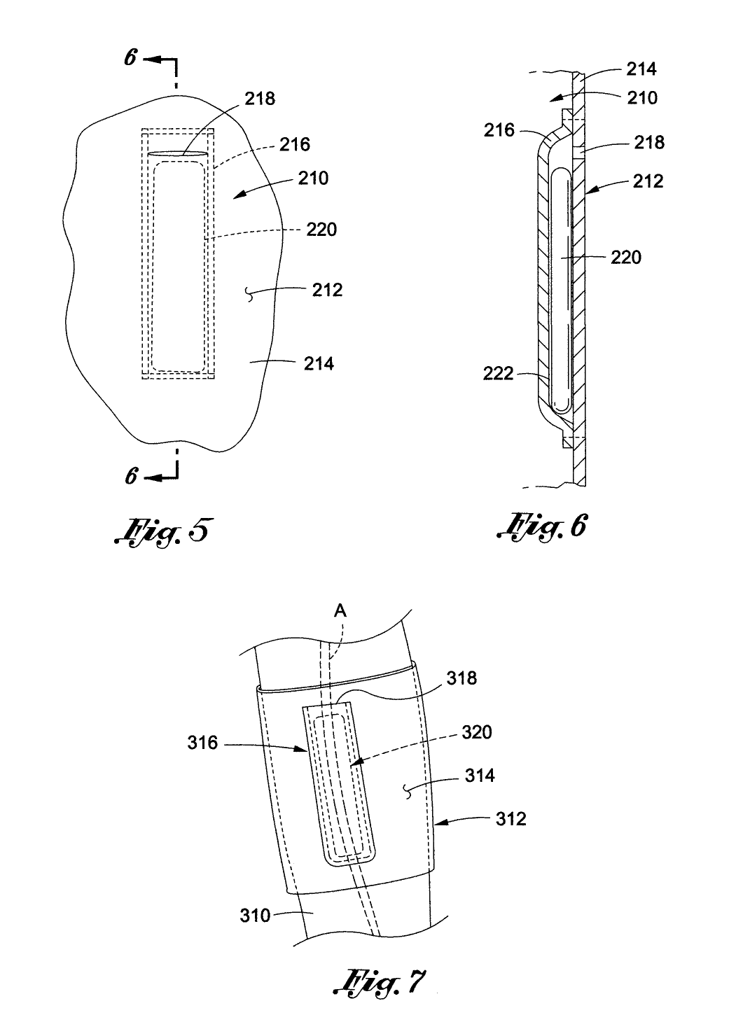

Turning to FIGS. 5 and 6 in conjunction to one another, yet another alternative embodiment of a thermal assistance garment is illustrated being worn by a user or wearer 210. Because the garment seen in FIGS. 5 and 6 is similar in many respects to that of FIGS. 1-3, features which are analogous in structure or function to those of FIGS. 1-3 are referenced on FIGS. 5 and 6 with the same numeral used above, but increased by two hundred (200).

Viewing FIGS. 5 and 6 in conjunction, a thermal assistance garment 212 embodying the present invention is illustrated. The garment 212 in this embodiment is configured as a pair of compression shorts similar in many respects to the embodiment of FIGS. 1-3. Accordingly, the garment 212 includes an elastic outer shell of material 214. The thermal assistance garment 212 according to this invention includes a pair of elongate generally vertically oriented pocket features, generally indicated with the arrowed numerals 216 (only one of which is seen in FIGS. 5 and 6). The pockets 216 are disposed in this embodiment on the inside of the shell material 214, and the shell material 214 outwardly defines merely an upper slit opening 218 opening inwardly to the pocket 216. An elongate thermal pack or package 220 is receivable inwardly via the slit opening 218 to be disposed inside of the garment 212 (i.e., inside of the shell material 214) and within a respective one of the pockets 216, as is indicated in FIGS. 5 and 6.

Importantly, as is seen in FIGS. 5 and 6, the pocket 216 is defined by material 222 which is selectively different from the material 214 from which the shell of the garment 212 is fashioned. The material 222 may be selected to have a heat transfer characteristic (i.e., thermal conductivity, or resistance value) which differs from the material 214. This feature of the embodiment of FIGS. 5 and 6 allows the selection of a material 222 for the pockets 216 which has a higher thermal conductivity than the shell material 214, if such is desirable. On the other hand, the pocket material 222 may also be selected to have a greater insulation value, thus mitigating (or slowing) the heat transfer between the user's core blood flow and the thermal packs 220

Turning now to FIG. 7 still another alternative embodiment of a thermal assistance device is illustrated. In this case, the device is configured as a band, strap, or wrap that may be worn on a wrist, arm, or leg, for example. Essentially, the device is configured as a flexible structure that may circumscribe and attach to a portion of the human body, such as a wrist, arm, leg, or foot, for example in order to provide a snug fit and mild compression. Because the device seen in FIG. 7 is similar in many respects to those seen in earlier Figures described above, features which are analogous in structure or function to those illustrated and described above are referenced on FIG. 7 with the same numeral used above, but increased by three hundred (300).

Viewing FIG. 7, a thermal assistance device 312 embodying the present invention is illustrated. The device 312 in this embodiment is configured as band, strap, or wrap which may be worn on or about a wrist, arm, leg, or foot, for example. The wearer's wrist, arm, leg, or foot is diagrammatically illustrated in FIG. 7, and is generally indicated in this Figure with the arrowed numeral 310. The device 312 includes a body portion 314 which may be elastic, although such is not essential to operability of the inventive device. That is, the body portion 314 may be configured as a wrap, possibly including hook-and-loop fastening material, which is simply wrapped about a person's arm, wrist, or leg in a selected location. The thermal assistance device 312 includes an elongate pocket, generally indicated with the arrowed numeral 316. The pocket 316 is disposed in this embodiment on the outside of the body 314 (although it may be disposed internally of the device, recalling FIGS. 5 and 6), and the pocket 316 defines an upper slit opening 318. As explained above, an elongate thermal pack or package 320 is receivable into the pocket 316 via the opening 318 to be disposed in abiding heat transfer relation with the skin of the person wearing device 312. As is seen in FIG. 7, the thermal pack 320 is disposed over the superficial exposure of an artery “A” such that an effective heat transfer relation is established between the thermal pack 320, the user's skin, and the underlying artery A. By effecting supplemental heating or cooling to the blood circulating in artery A, the device 312 is effective to assist in heating or cooling the person's core blood, or the blood supply to an extremity, such as a hand or foot.

Having considered the structure of several alternative embodiments of the thermal assistance garment herein disclosed, illustrated, and described, attention may now be directed more particularly to their method of operation. As will be appreciated viewing the drawing Figures described above, the present inventive garment disposes an elongate heat transfer pack or package 20, 120, 220, 320 over one or more selected superficially or near-superficially exposed arteries of the user, which arteries are conveying core blood, or conveying blood to an extremity. The thermal packs are maintained in good heat transfer relationship with the user's skin, and the thermal packs are elongate with their length aligned along the artery to which supplemental heating or cooling is to be provided.

Locations of the human body which are particularly favored for application of the present method are:
the femoral artery (upper thigh, inner groin);
the brachial artery (upper inner arm);
the ulnar/radial artery (inner wrist);
the posterior tibial artery (inner upper ankle);
the dorsalis pedis artery (top of the foot); and
the popliteal artery (back of the knee).

As will be appreciated by those familiar with human anatomy, the first two locations listed above are perhaps the most effective in supplying supplemental heating or cooling to the circulating core blood, although each of these locations can be of assistance in such an effort. The last four locations listed are perhaps of particular interest in providing supplemental heating to the blood supply to an extremity. The ulnar/radial artery is an especially effective location for supplying supplemental blood warming to the hands of a person who must work with high manual dexterity or hand strength in difficult conditions of extreme cold, or in conditions of rapid heat loss from the hands, such as immersion in cold water

Those skilled in the art will further appreciate that the present invention may be embodied in other specific forms without departing from the spirit or central attributes thereof. For example, the present invention may be embodied in a device or structure to be worn by a human but which is less than or only a portion of a garment, such as a wrist band or leg band, for example. Still alternatively, an arm sleeve or leg sleeve embodying the present invention could be utilized to hold thermal packs in selected locations of the human body consistently with the principles of this invention. Accordingly, because the foregoing description of the present invention discloses only particularly preferred exemplary embodiments of the invention, it is to be understood that other variations are recognized as being within the scope of the present invention. Accordingly, the present invention is not limited to the particular embodiment which has been described in detail herein. Rather, reference should be made to the appended claims to define the scope and content of the present invention.

I claim:

1. A thermal assistance device, said thermal assistance device comprising an especially configured holder for being disposed at a selected location of a wearer's body; said especially configured holder being configured for receiving and disposing a heating or cooling device in an abiding heat transfer relation with the wearer's skin, and in a resulting effective heat transfer relationship with the wearer's circulating blood in a superficially exposed or near-superficially exposed artery.

2. The thermal assistance device of claim 1 wherein said device has a configuration selected from the group consisting of: a band, a strap, and a wrap.

3. The thermal assistance device of claim 1 wherein said device has the configuration of a shirt.

4. The thermal assistance device of claim 3 wherein said shirt configured device includes sleeves, and said especially configured holder if configured as a pocket defined on at least one of said sleeves for receiving said heating or cooling device, said pocket being disposed at one of the ulnar/radial artery (inner wrist), and the brachial artery (upper inner arm) of the wearer.

5. The thermal assistance device shorts of claim 5 wherein said shirt configured device is fabricated to include elastic material such that said shirt configured device maintains compression on the body of the wearer.

6. The thermal assistance device of claim 1 wherein said device is configured as a shorts, said shorts having a pair of leg portions one for each of the wearer's left leg and right leg, and said holder if configured as a pair of pockets defined by said shorts one on each of the wearer's legs for receiving a pair of said heating or cooling devices, said pair of pockets being disposed over the femoral arteries of the wearer's legs.

7. The thermal assistance device shorts of claim 5 wherein said shorts is fabricated to include elastic material such that said shorts maintain compression on the body of the wearer.

8. The thermal assistance device of claim 1 wherein said especially configured holder is configured as a elongate pocket, and said heating or cooling device is also elongate, such that said thermal assistance device disposes said elongate heating or cooling device in abiding heat transfer relationship with the wearer's skin over and alignment along the length of said elongate pocket and elongate heating or cooling device with a superficial exposed artery of the wearer.

9. The thermal assistance device of claim 1 wherein said superficially exposed artery is selected from the group consisting of: the femoral artery (upper thigh, inner groin), the brachial artery (upper inner arm), the ulnar/radial artery (inner wrist), the posterior tibial artery (inner upper ankle), the dorsalis pedis artery (top of the foot), and the popliteal artery (back of the knee).

10. A thermal assistance garment, said thermal assistance garment being configured as a garment selected from the group consisting of: a shirt with sleeves, a shorts, and a band or strap or wrap for the wrist, arm, or leg; said thermal assistance garment further comprising an especially configured pocket disposed at a selected location of a wearer's body; said especially configured pocket being elongate and disposed over and in general alignment with a superficially exposed artery of the wearer's body, and said especially configured pocket being configured for receiving and disposing a heating or cooling device in an abiding heat transfer relation with the wearer's skin over and in general alignment with the superficially exposed artery; whereby a resulting effective heat transfer relationship is maintained between the heating or cooling device and the wearer's circulating blood in the superficially exposed artery.

11. The thermal assistance garment of claim 10 wherein said garment has a configuration selected from the group consisting of: a band, a strap, and a wrap.

12. The thermal assistance garment of claim 10 wherein said garment has a configuration selected from the group consisting of: a compression shirt and a compression shorts.

13. The thermal assistance device of claim 12 wherein said compression shirt configured thermal assistance garment includes sleeves, and said especially configured pocket is defined on at least one of said sleeves for receiving said heating or cooling device, said especially configured pocket being disposed at one of the ulnar/radial artery (inner wrist), and the brachial artery (upper inner arm) of the wearer.

14. The thermal assistance device garment of claim 12 wherein said shorts configured thermal assistance garment includes a pair of leg portions one for each of the wearer's left leg and right leg, and said shorts configured garment includes a pair of especially configured pockets one on each of said left leg and right leg of the wearer, said pair of especially configured pockets being for receiving a pair of said heating or cooling devices, said pair of especially configured pockets being disposed over the femoral arteries of the wearer's legs.

15. The thermal assistance garment of claim 10 wherein said especially configured pocket is elongate and is disposed over and in general alignment with a superficially exposed artery of the wearer, and said heating or cooling device is also elongate, such that said thermal assistance garment disposes said elongate heating or cooling device in abiding heat transfer relationship with the wearer's skin over and alignment along the length of said elongate pocket and elongate heating or cooling device with a superficial exposed artery of the wearer.

16. A method of providing thermal assistance to a human, said method comprising steps of: identifying a location on the user's body of a superficially exposed artery, providing a heating or cooling device, locating said heating or cooling device in abiding heat transfer relation with the user's skin over the location of the superficially exposed artery, and effecting heat transfer between the user's circulating blood in the superficially exposed artery and the heating or cooling device.

17. The method of claim 16 wherein said step of identifying a location on the user's body of a superficially exposed artery includes the step of selecting said location from the group including: the femoral artery (upper thigh, inner groin), the brachial artery (upper inner arm), the ulnar/radial artery (inner wrist), the posterior tibial artery (inner upper ankle), the dorsalis pedis artery (top of the foot), and the popliteal artery (back of the knee).

18. The method of claim 16 wherein said step of locating said heating or cooling device in abiding heat transfer relation with the user's skin over the identified location of a superficially exposed artery includes the step of providing a garment, band, strap or wrap configured to be worn by the user, and providing an especially configured holder on said garment, band, strap, or band for holding a heating or cooling device over and in heat transfer relation with the user's skin at the identified location, such that heat transfer is maintained between the user's circulating blood in the superficially exposed artery and the heating or cooling device.
